(19) United States
(12) Patent Application Publication
(10) Pub. No.: US 2026/0198865 A1
(43) Pub. Date: Jul. 16, 2026

(54) System and Method of Using DENSE Deep Networks to Predict Myocardial Strain

(71) Applicant: University of Virginia Patent Foundation, Charlottesville, VA (US)

(72) Inventors: Miaomiao Zhang, Charlottesville, VA (US); Frederick H. Epstein, Charlottesville, VA (US); Pengcheng Lei, Charlottesville, VA (US); Sona Ghadimi, Charlottesville, VA (US)

(21) Appl. No.: 19/452,217

(22) Filed: Jan. 16, 2026

Related U.S. Application Data
(60) Provisional application No. 63/746,129, filed on Jan. 16, 2025.

(51) Int. Cl. A61B 5/00; A61B 5/055; G01R 33/56; G06T 7/00; G06T 7/246; G06T 7/38; G16H 30/40

(52) U.S. Cl. CPC A61B 5/7267; A61B 5/0044; A61B 5/055; G01R 33/5608; G06T 7/0012; G06T 7/246; G06T 7/38; G16H 30/40; A61B 2576/023; G06T 2207/10016; G06T 2207/10088; G06T 2207/20081; G06T 2207/20084; G06T 2207/30048

(57) ABSTRACT

A neural network predicts a sequence of myocardial motions from cardiac image data of a heart of a subject. DENSE cardiac magnetic resonance contour videos of cine data are used as input, and the output provides predicted displacement fields, supervised by the DENSE displacements. In the testing phase, DENSE data are no longer required; the trained network takes standard cine CMR bSSFP contour videos as input and predicts the final displacements.

### CROSS-REFERENCE TO RELATED APPLICATIONS

This application claims priority to co-pending U.S. Provisional Patent App. Ser. No. 63/746,129, filed on Jan. 16, 2025, and entitled System and Method for Improved DENSE-Guided Deep Networks to Predict Myocardial Strain from Routine Cine Magnetic Resonance Images, which is incorporated in its entirety as if set forth fully herein.

### STATEMENT OF RIGHTS UNDER FEDERALLY-SPONSORED RESEARCH

This invention was made with government support under Grant No. 1R21EB032597, awarded by the National Institutes of Health. The government has certain rights in the invention.

### BACKGROUND

Cardiac magnetic resonance (CMR) cine imaging is widely regarded as the gold-standard technique for the non-invasive assessment of cardiac function. The term “cine” include series of MR frames of data that be viewed as a kind of video or replayed sequence of frames. Typically, images are acquired using a breath-held 2D segmented electrocardiography-gated (ECG-gated) balanced steady-state free precession (bSSFP) pulse sequence. Prospective or retrospective ECG-gating is used to synchronize segmented data acquisition to the cardiac cycle over multiple heartbeats during a breath-hold to generate images at multiple cardiac phases across the cardiac cycle.

The approach of using electrocardiography (ECG) triggering and breath-hold acquisition has several limitations. Firstly, the ECG signal can be distorted due to the magnetohydrodynamic effect [1], rapid switch of magnetic gradients [2], as well as radiofrequency interference [3,4] resulting in mis-triggering. This distortion is worse at higher field strengths such as 3T. Furthermore, placement of the ECG leads requires expertise and increases the time to prepare the patient for the CMR exam. Additionally, a significant number of patients are not able to adequately hold their breath during cine acquisition, resulting in motion artifacts and the need to repeat image acquisition of the same slice location on subsequent breath-holds. Even if the patient can perform good breath-holds, this approach is inefficient, as it requires 10-12 breath-holds to cover the left ventricle (LV) and requires coordination between the operator and the patient.

To partially alleviate issues caused by cardiac and respiratory motion, the conventional solutions can be separated into 3 categories: navigator-based methods, real-time methods, and self-gating methods. Navigator-echo based methods which accept or reject data based on the position of the diaphragm have been used to account for respiratory motion [5-8]. However, the usage of diaphragmatic navigators typically precludes retrospective ECG gating, and the total scanning time is prolonged depending on respiratory gating efficiency. More recently, projection navigators acquired during steady state free precession (SSFP) have been utilized to perform respiratory tracking without the need for a separate diaphragmatic navigator

, but this approach is still limited by navigator gated efficiency. These methods may account for respiratory motion, but they do not provide a mechanism to account for cardiac motion.

Other “self-navigated” techniques [10, 11] have been proposed to eliminate the need for ECG synchronization by acquiring and processing additional magnetic resonance (MR) signals to derive cardiac cycle timing information. Relative to ECG-gated techniques, methods that use extra lines of data to acquire the self-gating signals result in decreased imaging efficiency. In the clinical setting, when the ECG or breath-holds do not perform adequately, operators may resort to real-time imaging techniques. Although these real-time methods do not require ECG gating, they have been shown to sacrifice spatial and/or temporal resolution [12-14].

Cine DENSE (displacement encoding with stimulated echoes) is a myocardial strain imaging technique that typically requires breath-holding during image acquisition. Subtraction of phase-cycled data can be utilized to suppress the artifact-generating T1-relaxation echo [15, 16]. With free-breathing, suppression of the T1-relaxation echo may not effective, however, due to respiratory motion between the phase-cycled data, resulting in artifacts.

Further described, cine DENSE [15] is a technique that is accurate [17], reproducible for both global and regional measurements [18], and amenable to rapid displacement and strain analysis [19]. With these properties, it can have many clinical applications. For example, Auger et al. [20] recently showed in heart failure patients that cine DENSE can quantify late mechanical activation and predict treatment response. Mangion et al

showed the prognostic value of cine DENSE in acute myocardial infarction, and Jing et al

showed the detection of subclinical contractile dysfunction in childhood obesity. Like many cardiac MRI acquisitions, cine DENSE is generally performed during breath-holding. However, in patient populations such as heart failure, acute myocardial infarction, children, and others, multiple breath-holds can be taxing. In addition, performing multi-breath-hold protocols can be complex for technologists.

While diaphragm-based navigator (dNAV) methods [23], including those for cine DENSE [16], are available, a paradigm shift is occurring in cardiac MRI where self-navigation with motion estimation and motion correction is superseding dNAV-based methods for free-breathing acquisitions. Self-navigated methods have the advantages that they do not require complex dNAV setup procedures and they can be more efficient than dNAVs because, with motion estimation and correction, they use data acquired during much or all of the respiratory cycle, whereas in dNAV-based methods data acquisition is restricted to a narrow band of the respiratory cycle. Self-navigated techniques have previously been developed for multiple cardiac MRI applications including cine imaging [24-26], late-gadolinium-enhanced imaging [27], coronary artery imaging [28-30], and T1 mapping [31], however they have not yet been developed for strain imaging methods such as cine DENSE. For cine DENSE imaging, two echoes are generally present in the acquisition window [15], namely the desired displacement-encoded stimulated echo and an echo due to T1 relaxation that can cause image artifacts. A two-point phase cycling method is typically used to suppress the T1-relaxation echo, wherein two acquisitions comprised of stimulated echoes of opposite signs and T1-relaxation echoes of the same sign are subtracted [15]. While phase cycling (used in conjunction with through-plane dephasing [32]) effectively suppresses the T1-relaxation echo for breath-hold DENSE protocols, this subtraction-based method can be ineffective for free-breathing scans because phase-cycled interleaves may be acquired at different respiratory positions where tissues contribute differently to the T1-relaxation signals. Insufficient suppression of the T1-relaxation echo leads to striping artifacts [18], which represents a unique and major challenge for free-breathing self-navigated cine DENSE. In addition to creating challenges for suppression of the T1-relaxation echo, respiratory motion induces blurring of the stimulated-echo image, as it does for other MR images.

It is with respect to these and other considerations that the various aspects of the disclosed technology as described below are presented. For example, Cine displacement encoding with stimulated echoes (DENSE) MRI [15] is well-established and dedicated strain imaging technique. Recent studies demonstrated the potential of cine DENSE for detection of subclinical myocardium dysfunction and patient treatment stratification [16-18]. Cine DENSE acquisition is typically performed during breath-holding and multiple breath-holds are required per exam [15-19]. However, such protocols can be challenging in patient populations such as heart failure, pediatrics, and others [19]). In practice, imperfect breath-holds lead to repetitions of acquisitions and reduce imaging efficiency. A reliable free-breathing method can overcome these challenges.

Among the various techniques for free-breathing cardiac MRI, diaphragm-based navigator (dNAV) [20] was implemented for cine DENSE and was able to reduce breathing artifacts [21, 22]. However, the dNAV method requires extra scout scans and often results in variable imaging quality and efficiency [23-25]. A better solution for free-breathing cardiac MRI is self-navigation where the respiration information is extracted from the imaging data itself and used for motion compensation. Such methods have been developed for cardiac MRI applications such as SSFP cine imaging [26-29], angiography [30, 31], and T1 mapping [32].

In a previous study, a self-navigated reconstruction framework for free-breathing cine DENSE was developed [33]. The method addressed two major types of artifacts, namely the striping and blurring artifacts due to inter-heartbeat respiratory motion [33]. Cine DENSE imaging signal contains two echoes, the displacement-encoded stimulated echo and the artifact-generating T1-relaxation echo [15]. Typically, two phase-cycled datasets during different heartbeats are acquired and subtracted to suppress the T1-relaxation echo [34]. With free-breathing, the suppression is not effective with the subtraction, which leads to striping artifacts. Phantom and in vivo experiments demonstrated that the residual energy of the T1-relaxation echo (rT1E) after phase-cycling subtraction increased as the motion between the phase-cycled datasets increased. Minimal rT1E of the post-subtraction data identified phase-cycling pairs that were acquired at similar respiratory positions and reduced striping artifacts. After subtraction of the matched phase-cycling pairs, stimulated-echo only image-based navigators (ste-iNAVs) were reconstructed from the post-subtraction k-space data. In-plane motion due to respiration was then estimated with the ste-iNAVs and corrected to reduce blurring.

However, this reconstruction framework still had a few drawbacks. The image quality was not guaranteed and the imaging efficiency was not optimized. The reconstruction was performed retrospectively after the data acquisition was completed with a prescribed protocol and fixed acquisition order. DENSE data were acquired with three repetitions to provide multiple candidates of phase-cycling pairs and a better chance of suppressing the T1-relaxation echo sufficiently rather than acquiring each phase-cycling just once. Yet, such a protocol cannot guarantee high-quality free-breathing cine DENSE as the number of repetitions necessary may vary from subject to subject. Increasing the repetition number increases the possibility of matching phase-cycling data for every k-space segment but reduces imaging efficiency. Using real-time feedback on rT1E to guide data acquisition can potentially guarantee sufficient suppression of the T1-relaxation echo without sacrificing the imaging efficiency.

In addition to blurring, respiratory motion within each heartbeat (intra-heartbeat motion) could induce phase errors in the stimulated-echoes of the prior art. In cine DENSE, tissue motion is encoded into the phase of the stimulated-echoes. The motion-related phase is linear with the displacement of the tissue that happens between application of the preparation pulses and the k-space data acquisition. Along with the myocardial displacement with the heart contracting and relaxing periodically, the bulk movement of the heart due to respiration is also encoded into the stimulated-echo signal.

Prior efforts to improve CMR also use convolutional neural networks, such as U-Net

to improve results. Processes discussed herein take advantage of image analysis by use of convolutional neural networks (CNNs). CNNs are multi-layer feed-forward networks specifically designed to recognize features in image data. A typical application of CNNs consists of recognition of various objects in images. However convolutional networks have been successfully used for various different tasks, too. The neurons in CNNs work by considering a small portion of the image, referred to herein as a patch. The patches are inspected for features that can be recognized by the network. As a simple example, a feature may be a vertical line, an arch, or a circle. These features are then captured by the respective feature maps of the network. A combination of features is then used to classify the image, or in an example case, each pixel.

3D UNet was originally proposed by Cicek et al. [38] for automatic segmentation of Xenopus (a highly aquatic frog) kidney. It has an encoder-decoder style architecture with skip connections between corresponding layers in encoding and decoding paths. This architecture is very popular for medical image segmentation. All the deep learning models used in this study have the same architecture, the 3D UNet. 3D in the name indicates that the input to this network is a 3D image. UNet refers to the structure of the network, which resembles the letter ‘U’. FIG. 3 shows the block representation of 3D UNet architecture.

Each convolutional block has two convolutions followed by max pooling. Every convolution is immediately followed by a rectified linear unit (ReLU) activation and batch normalization layer. Each deconvolutional block consists of two convolutions followed by a deconvolution to regain spatial dimension. Moreover, there are skip connections from the encoding path to decoding path at corresponding spatial dimensions. These are shown by green arrows. The very final convolution (shown by an arrow) that generates a three-dimensional feature map is followed by a softmax activation in order to obtain a pseudo-random probability distribution at each pixel representing its class membership. A U-Net is one example of a convolutional neural network and is not limiting of this disclosure.

This disclosure addresses a need in the art for using DENSE techniques, along with convolutional neural networks (CNNs) to capture large rotational motion of the myocardium associated with twist and torsion over time while addressing previous drawbacks of earlier systems.

### SUMMARY

Other aspects and features according to the example embodiments of the disclosed technology will become apparent to those of ordinary skill in the art, upon reviewing the following detailed description in conjunction with the accompanying figures.

In one embodiment, a computer implemented method trains a neural network to predict a sequence of myocardial motions from cardiac image data of a heart of a subject. The method includes using a computer with computer memory connected to a processor to execute software stored in the computer memory and executing computerized steps. The steps include acquiring a time series of cardiac magnetic resonance (CMR) contour videos of cine data formatted according to displacement encoding with stimulated echoes (DENSE); processing the cine data with a rotation estimation sub-network that models rotational dynamics of a left ventricle of the heart and storing a rotation output in the computer memory; processing the cine data with a radial motion prediction sub-network that performs deformable image registration on the cine data and stores a registration output in the computer memory; applying the rotation output and the registration output to a fusion network that predicts myocardial displacements present in the cine data; and supervising the fusion network with ground truth DENSE data to learn the myocardial displacements for diverse sets of cine data.

### BRIEF DESCRIPTION OF THE DRAWINGS

Reference will now be made to the accompanying drawings, which are not necessarily drawn to scale.

FIG. 1A is a diagram illustrating a magnetic resonance imaging (MRI) system capable of implementing certain aspects of the disclosed technology in accordance with one or more embodiments.

FIG. 1B depicts a computer environment in which the embodiments of this disclosure may operate.

FIG. 2A is a PRIOR ART timing diagram for the 3D cine DENSE pulse sequence. A navigator echo is played out before the ECG trigger so as not to interfere with displacement encoding prior to or imaging during the onset of myocardial contraction (marked as the NAV block in the diagram). Immediately after detection of the R wave, a spectrally-selective fat suppression pulse is applied (marked as the FS block in the diagram), which is followed by the displacement encoding module. A segmented data acquisition, which includes displacement-unencoding gradients and a 3D stack-of-spirals k-space trajectory, is used to sample the displacement encoded longitudinal magnetization at multiple cardiac phases. In this diagram, the displacement-encoding gradients are applied in the frequency-encoding direction (the gradient waveform shown in the solid line on the GFE axis). However, more generally, displacement encoding can be applied in any direction by also applying the gradients shown in dashed lines on the GPE and/or GSS axes, respectively. Applying gradients in multiple directions is employed for balanced multi-point displacement encoding. In practice, to minimize the echo time, the displacement-unencoding gradients are combined with spatial encoding gradients, but are shown separately in this diagram for clarity.

FIG. 2B shows a PRIOR ART diagram of a pulse sequence used for free-breathing cine DENSE. Fat saturation (FS) is applied after each ECG trigger, followed by displacement encoding (DE) pulses, spiral acquisitions (ACQ) are set forth herein as respective frames or images that each have phase cycled interleaves. A series of frames or images collectively form a segment of image data between heart beats shown as the ECG triggers. The series of images or frames and the diaphragm navigator (dNAV). Localized generation of stimulated echoes is performed by applying slice selection for all RF pulses, including those in the displacement encoding module. Data acquisition uses a segmented spiral trajectory with golden angle rotation of the trajectory through cardiac frames. Each iNAV frame consists of four cine DENSE frames (i.e., eight consecutive spiral interleaves).

FIG. 3 illustrates a PRIOR ART three dimensional (3D) U-Net Architecture that provides additional background for the embodiments disclosed herein.

FIG. 4 shows an overview of a proposed network to predict DENSE displacement from input cine bSSFP images. During network training, DENSE CMR contours are used as input, and the output consists of predicted displacement fields, supervised by the DENSE displacements. In the testing phase, DENSE data are no longer required; the trained network takes standard cine CMR bSSFP contour videos as input and predicts the final displacements.

FIG. 5 shows a boxplot of absolute segmental circumferential strain errors from the model and three prior models on standard bSSFP cine CMR tests with six segments.

FIG. 6 shows examples of strain analysis of bSSFP CMRs using network-predicted myocardial displacements from previous models and the model of this disclosure, compared with DENSE-strain. From top to bottom: visualizations of segmental circumferential strain-time curves, strain-time maps, circumferential strain maps, and estimated rotational movements.

FIG. 7 illustrates equations referenced in this disclosure by number.

### DETAILED DESCRIPTION

In some aspects, the disclosed technology relates to free-breathing cine DENSE (displacement encoding with stimulated echoes) imaging. Although example embodiments of the disclosed technology are explained in detail herein, it is to be understood that other embodiments are contemplated. Accordingly, it is not intended that the disclosed technology be limited in its scope to the details of construction, and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology.

Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, referred to herein as an “area of interest” or a “region of interest.”

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the n^{th} reference in the list. For example, [3] refers to the 3^{rd} reference in the list, namely Young, et al. All references cited and discussed in this specification are incorporated herein by reference in their entireties and to the same extent as if each reference was individually incorporated by reference.

A detailed description of aspects of the disclosed technology, in accordance with various example embodiments, will now be provided with reference to the accompanying drawings. The drawings form a part hereof and show, by way of illustration, specific embodiments and examples. In referring to the drawings, like numerals represent like elements throughout the several figures. The following description includes discussion of some example implementations and corresponding results. Some experimental data are presented herein for purposes of illustration and should not be construed as limiting the scope of the disclosed technology in any way or excluding any alternative or additional embodiments.

### Example MRI System

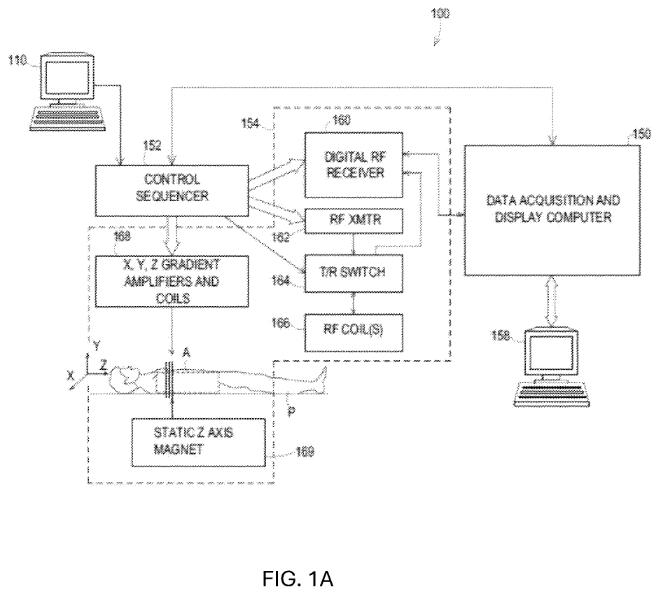

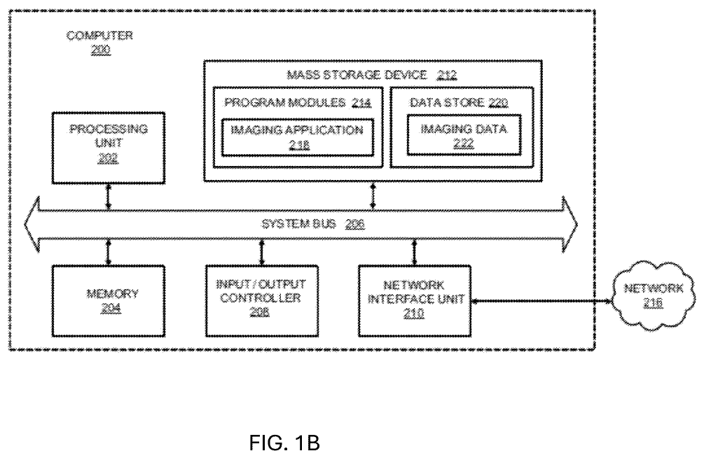

FIG. 1A is a system diagram illustrating an example of a magnetic resonance imaging (MRI) system 100, including a data acquisition and display computer 150 coupled to an operator console 110, an MRI real-time control sequencer 152, and an MRI subsystem 154. The MRI subsystem 154 may include XYZ magnetic gradient coils and associated amplifiers 168, a static Z-axis magnet 169, a digital RF transmitter 162, a digital RF receiver 160, a transmit/receive switch 164, and RF coil(s) 166. The MRI subsystem 154 may be controlled in real time by control sequencer 152 to generate magnetic and radio frequency fields that stimulate magnetic resonance phenomena in a subject (patient) P to be imaged, for example, to implement magnetic resonance imaging sequences in accordance with various example embodiments of the disclosed technology described herein. An image of an area of interest A of the subject P (which may also be referred to herein as a “region of interest”) may be shown on display 158. The display 158 may be implemented through a variety of output interfaces, including a monitor, printer, or data storage.

The area of interest A corresponds to a region associated with one or more physiological activities in subject P. The area of interest shown in the example embodiments of FIG. 1A corresponds to a chest region of subject P, but it should be appreciated that the area of interest for purposes of implementing various aspects of the disclosure presented herein is not limited to the chest area. It should be recognized and appreciated that the area of interest in various embodiments may encompass various areas of subject P associated with various physiological characteristics, such as, but not limited to the heart region. Physiological activities that may be evaluated by methods and systems in accordance with various embodiments of the disclosed technology may include, but are not limited to, cardiac activity and conditions. It should be appreciated that any number and type of computer-based medical imaging systems or components, including various types of commercially available medical imaging systems and components, may be used to practice certain aspects of the disclosed technology. Systems as described herein with respect to example embodiments are not intended to be specifically limited to magnetic resonance imaging (MRI) implementations or the particular system shown in FIG. 1A.

One or more data acquisition or data collection steps as described herein in accordance with one or more embodiments may include acquiring, collecting, receiving, or otherwise obtaining data such as imaging data corresponding to an area of interest. By way of example, data acquisition or collection may include acquiring data via a data acquisition device, receiving data from an on-site or off-site data acquisition device or from another data collection, storage, or processing device. Similarly, data acquisition or data collection devices of a system in accordance with one or more embodiments of the disclosed technology may include any device configured to acquire, collect, or otherwise obtain data, or to receive data from a data acquisition device within the system, an independent data acquisition device located on-site or off-site, or another data collection, storage, or processing device.

It should be appreciated that any number and type of computer-based medical imaging systems or components, including various types of commercially available medical imaging systems and components, may be used to practice certain aspects of the disclosed technology. Systems as described herein with respect to example embodiments are not intended to be specifically limited to magnetic resonance imaging (MRI) implementations or the particular system shown in FIG. 1A.

One or more data acquisition or data collection steps as described herein in accordance with one or more embodiments may include acquiring, collecting, receiving, or otherwise obtaining data such as imaging data corresponding to an area of interest. By way of example, data acquisition or collection may include acquiring data via a data acquisition device, receiving data from an on-site or off-site data acquisition device or from another data collection, storage, or processing device. Similarly, data acquisition or data collection devices of a system in accordance with one or more embodiments of the disclosed technology may include any device configured to acquire, collect, or otherwise obtain data, or to receive data from a data acquisition device within the system, an independent data acquisition device located on-site or off-site, or another data collection, storage, or processing device.

### Example Computing System

FIG. 1B is a computer architecture diagram showing a general computing system capable of implementing aspects of the disclosed technology in accordance with one or more embodiments described herein. A computer 200 may be configured to perform one or more functions associated with embodiments illustrated in one or more of the figures. For example, the computer 200 may be configured to perform various aspects of free-breathing parameter mapping with high-contrast image registration in accordance with example embodiments, such as magnetic resonance imaging data acquisition, image registration, and calculating parameter maps. It should be appreciated that the computer 200 may be implemented within a single computing device or a computing system formed with multiple connected computing devices. The computer 200 may be configured to perform various distributed computing tasks, in which processing and/or storage resources may be distributed among the multiple devices. The data acquisition and display computer 150 and/or operator console 110 of the system shown in FIG. 1B may include one or more systems and components of the computer 200.

As shown, the computer 200 includes a processing unit 202 (“CPU”), a system memory 204, and a system bus 206 that couples the memory 204 to the CPU 202. The computer 200 further includes a mass storage device 212 for storing program modules 214. The program modules 214 may be operable to perform associated with embodiments illustrated in one or more of the figures herein. The program modules 214 may include an imaging application 218 for performing data acquisition and/or processing functions as described herein, for example to acquire and/or process image data corresponding to magnetic resonance imaging of an area of interest. The computer 200 can include a data store 220 for storing data that may include imaging-related data 222 such as acquired data from the implementation of magnetic resonance imaging in accordance with various embodiments of the disclosed technology.

The mass storage device 212 is connected to the CPU 202 through a mass storage controller (not shown) connected to the bus 206. The mass storage device 212 and its associated computer-storage media provide non-volatile storage for the computer 200. Although the description of computer-storage media contained herein refers to a mass storage device, such as a hard disk or CD-ROM drive, it should be appreciated by those skilled in the art that computer-storage media can be any available computer storage media that can be accessed by the computer 200.

By way of example and not limitation, computer storage media (also referred to herein as “computer-readable storage medium” or “computer-readable storage media”) may include volatile and non-volatile, removable and non-removable media implemented in any method or technology for storage of information such as computer-storage instructions, data structures, program modules, or other data. For example, computer storage media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer 200. “Computer storage media”, “computer-readable storage medium” or “computer-readable storage media” as described herein do not include transitory signals.

According to various embodiments, the computer 200 may operate in a networked environment using connections to other local or remote computers through a network 216 via a network interface unit 210 connected to the bus 206. The network interface unit 210 may facilitate connection of the computing device inputs and outputs to one or more suitable networks and/or connections such as a local area network (LAN), a wide area network (WAN), the Internet, a cellular network, a radio frequency (RF) network, a Bluetooth-enabled network, a Wi-Fi enabled network, a satellite-based network, or other wired and/or wireless networks for communication with external devices and/or systems. The computer 200 may also include an input/output controller 208 for receiving and processing input from any of a number of input devices. Input devices may include one or more of keyboards, mice, stylus, touchscreens, microphones, audio capturing devices, and image/video capturing devices. An end user may utilize the input devices to interact with a user interface, for example a graphical user interface, for managing various functions performed by the computer 200. The bus 206 may enable the processing unit 202 to read code and/or data to/from the mass storage device 212 or other computer-storage media. The computer-storage media may represent apparatus in the form of storage elements that are implemented using any suitable technology, including but not limited to semiconductors, magnetic materials, optics, or the like. The computer-storage media may represent memory components, whether characterized as RAM, ROM, flash, or other types of technology.

The computer storage media may also represent secondary storage, whether implemented as hard drives or otherwise. Hard drive implementations may be characterized as solid state, or may include rotating media storing magnetically-encoded information. The program modules 214, which include the imaging application 218, may include instructions that, when loaded into the processing unit 202 and executed, cause the computer 200 to provide functions associated with one or more example embodiments and implementations illustrated in the figures. The program modules 214 may also provide various tools or techniques by which the computer 200 may participate within the overall systems or operating environments using the components, flows, and data structures discussed throughout this description.

In general, the program modules 214 may, when loaded into the processing unit 202 and executed, transform the processing unit 202 and the overall computer 200 from a general-purpose computing system into a special-purpose computing system. The processing unit 202 may be constructed from any number of transistors or other discrete circuit elements, which may individually or collectively assume any number of states. More specifically, the processing unit 202 may operate as a finite-state machine, in response to executable instructions contained within the program modules 214. These computer-executable instructions may transform the processing unit 202 by specifying how the processing unit 202 transitions between states, thereby transforming the transistors or other discrete hardware elements constituting the processing unit 202. Encoding the program modules 214 may also transform the physical structure of the computer-storage media. The specific transformation of physical structure may depend on various factors, in different implementations of this description. Examples of such factors may include, but are not limited to, the technology used to implement the computer-storage media, whether the computer storage media are characterized as primary or secondary storage, and the like. For example, if the computer storage media are implemented as semiconductor-based memory, the program modules 214 may transform the physical state of the semiconductor memory, when the software is encoded therein. For example, the program modules 214 may transform the state of transistors, capacitors, or other discrete circuit elements constituting the semiconductor memory.

As another example, the computer storage media may be implemented using magnetic or optical technology. In such implementations, the program modules 214 may transform the physical state of magnetic or optical media, when the software is encoded therein. These transformations may include altering the magnetic characteristics of particular locations within given magnetic media. These transformations may also include altering the physical features or characteristics of particular locations within given optical media, to change the optical characteristics of those locations. Other transformations of physical media are possible without departing from the scope of the present description, with the foregoing examples provided only to facilitate this discussion.

### DENSE Pulse Sequence

Cine DENSE techniques have been described in reference [2] as encoding “tissue displacement (typically relative to an end-diastolic reference time) into the phase of the MR image. Since displacement is encoded in the phase, the estimation of tissue motion does not require tag detection, and displacement and strain analysis is less time-consuming for DENSE than for conventional tagging. Previously, 2D cine DENSE has been described and validated using 2D myocardial tagging as a reference standard.” (citations omitted). The same reference

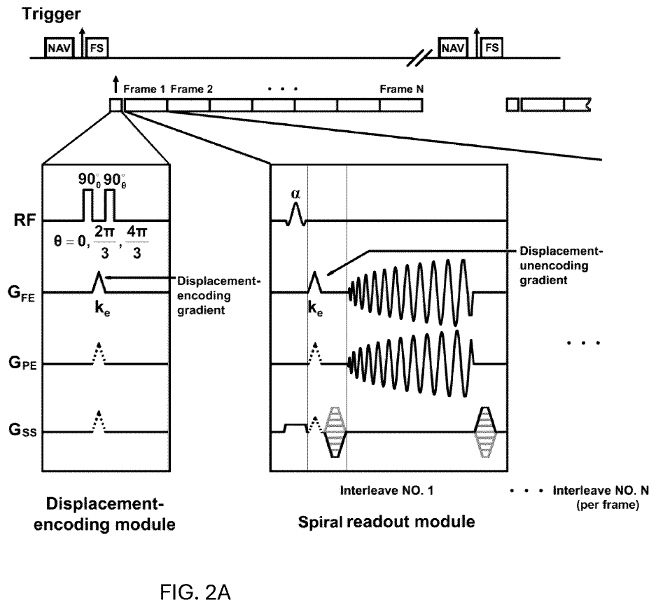

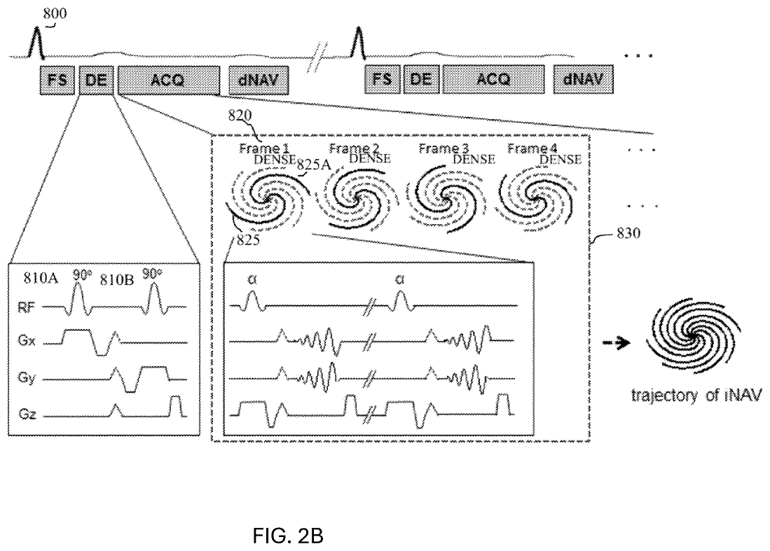

discusses minimizing echo time (TE) and increase signal-to-noise ratio (SNR) compared to an echoplanar approach, data were sampled using a spiral k-space trajectory. The specific design details of the sequence from this early use of DENSE are shown in FIG. 2A taken from reference [2].

To experimentally investigate free-breathing cine DENSE imaging using the matchmaking framework with motion compensation, a previously-described spiral cine DENSE pulse sequence [2], was modified. The previously-described spiral cine DENSE pulse sequence supports two-point phase-cycling, to include golden angle rotation [23, 24] through time frames within the cardiac cycle and localized generation of stimulated echoes, as shown in FIG. 2. Golden angle (α) rotation facilitates the reconstruction of iNAVs by utilizing consecutively acquired interleaves. In the current design, each fully sampled iNAV frame consisted of eight consecutively acquired interleaves, i.e. four consecutive frames with golden angle rotation between them and two interleaves per frame with uniform rotation. Localized generation of stimulated echoes was implemented by applying slice selection for all RF pulses that contribute to the stimulated echo such that stimulated echoes were generated only in the region where the RF pulse profiles intersect [19, 25]. It is noted that, with this pulse sequence, while the stimulated echo is localized to the heart region, the T1-relaxtion echo originates from the entire slice. The cine DENSE pulse sequence also supported acquisition of a dNAV at end-diastole, as previously described [2]. All DENSE scans employed multi-coil acquisitions, and multi-channel data were adaptively combined to reconstruct complex images with sensitivity maps estimated 15 from the data itself, as previously described [26].

For background, FIG. 2A is taken from Ref. [2] and shows a navigator echo was played out before the ECG trigger so as not to interfere with displacement encoding prior to or imaging during the onset of myocardial contraction (marked as the NAV block in the diagram). Immediately after detection of the R wave, a spectrally-selective fat suppression pulse is applied (marked as the FS block in the diagram), which is followed by the displacement encoding module. A segmented data acquisition, which includes displacement-unencoding gradients and a 3D stack-of-spirals k-space trajectory, is used to sample the displacement encoded longitudinal magnetization at multiple cardiac phases. In this diagram, the displacement-encoding gradients are applied in the frequency-encoding direction (the gradient waveform shown in the solid line on the GFE axis). However, more generally, displacement encoding can be applied in any direction by also applying the gradients shown in dashed lines on the GPE and/or GSS axes, respectively. Applying gradients in multiple directions is employed for balanced multi-point displacement encoding. In practice, to minimize the echo time, the displacement-unencoding gradients are combined with spatial encoding gradients, but are shown separately in this diagram for clarity.

FIG. 2B is from U.S. Pat. No. 10,830,855 , which is incorporated herein by reference as if set forth fully in this disclosure. FIG. 2B shows a diagram of an example pulse sequence that was previously used for free-breathing cine DENSE. Fat saturation (FS) is applied after each ECG trigger (800), followed by displacement encoding (DE) pulses (810A, 810B). Spiral acquisitions (ACQ) are set forth in FIG. 2B as respective frames or images (820) that each have phase cycled interleaves (825A, 825B). A series of frames or images (820) collectively form a segment (830) of image data between heart beats shown as the ECG triggers (800), along with the series of images or frames (820) and the diaphragm navigator (dNAV). Localized generation of stimulated echoes is performed by applying slice selection for all RF pulses, including those in the displacement encoding module. Data acquisition uses a segmented spiral trajectory with golden angle rotation of the trajectory through cardiac frames. Each iNAV frame consists of four cine DENSE frames (i.e., eight consecutive spiral interleaves).

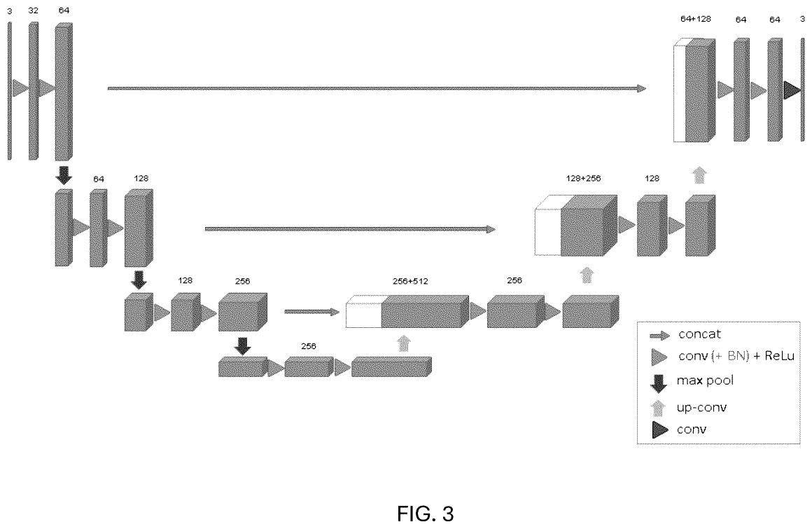

With the backdrop of FIGS. 2A and 2B for DENSE techniques, along with FIG. 3 showing the basic structure of a U-Net convolutional neural network, this disclosure expands the subject matter to allow for the technology to be used in to capture large rotational motion of the myocardium associated with twist and torsion over time.

Myocardial strain imaging provides a valuable tool for detecting subclinical left ventricular (LV) dysfunction and adding prognostic value in assessing various types of heart disease. Recent studies have utilized highly accurate strain-dedicated techniques, such as displacement encoding with stimulated echoes (DENSE), to train a deep learning (DL) framework to predict the myocardial displacements/deformations from routine cine balanced steady state free precession (bSSFP) images. However, these methods have shown limited performance in capturing the large rotational motion of the myocardium associated with twist and torsion over time, which are important aspects of myocardial mechanics. To address this gap, this paper introduces a novel DENSE-guided DL network that explicitly accounts for large rotational motion to further improve strain analysis of standard cine bSSFP images 402.

Specifically, a proposed network 400 includes two components: (i) a time-series rotation estimation sub-network 405 employing a 3D convolutional encoder-decoder architecture 410 to model the large rotational dynamics of the left ventricle (LV) myocardium over time, and (ii) a radial motion prediction sub-network 425 based on deformable image registration 430. The output of these two sub-networks 400, 425 was integrated and refined through a fusion network 450 to predict the final myocardial displacements, supervised by DENSE ground truth. Experimental results show that the method disclosed herein improves the accuracy of myocardial strain with effectively captured large rotations.

Myocardial strain assessment based on cine bSSFP CMR images plays an important role in optimizing cardiac resynchronization therapy treatment planning [39, 40], predicting outcomes post-myocardial infarction [41], and identifying subclinical cardiac dysfunction in obesity and diabetes [42]. Feature tracking (FT), a commonly used software for analyzing myocardial strain from cine CMR images in clinical practice, is designed to capture myocardial motion and deformation over time [43, 44]. While FT is convenient and easily integrated into clinical workflows, its accuracy in motion estimation is often limited due to image noise and artifacts. [45].

Deep learning (DL) has shown superior performance in predicting myocardial motion and strain from cine CMRs. However, most existing methods use unsupervised learning or suboptimal training data, yielding unsatisfactory results [43]. Recent research publications [40, 46, 47] have leveraged advanced imaging techniques, such as displacement encoding with stimulated echoes (DENSE), to supervise network learning for motion prediction from cine CMRs. In particular, the first DENSE guided deep network was proposed to predict myocardial options from segmented contour videos of myocardium [46], demonstrating improved accuracy of strain analysis. Inspired by this work [46], Xing et al. at reference [40] developed a joint framework that estimated accurate strain maps from the latent space of motion features to benefit the downstream task of late mechanical activation detection. While significant progress has been made, these methods lack an explicit modeling of large rotational motions, which are important features for detecting myocardial dysfunction (e.g., torsion and twist) [48-50].

Despite that DENSE CMR was shown to effectively capture a large degree of rotational movements [51], current DL networks tend to under-predict this factor, limiting the accuracy of subsequent strain analysis [40, 46, 43]. To address this issue, this disclosure develops a DENSE-guided deep motion network 400 that explicitly models large rotations for an improved myocardial strain analysis from standard cine CMRs. More specifically, non-limiting embodiments will introduce a time-series rotation estimation network 405, supervised by the tangential component of decomposed DENSE ground truth displacements as set forth below.

This network 400 is designed to accurately predict large pixel-wise rotations from cine CMR images 402. This disclosure will simultaneously train this network 400 with a deformable registration network 430 designed to effectively capture the radial components of myocardial motion, accommodating minor rotational variations. The outputs of these two sub-networks are then integrated through a data fusion module 450 to predict the final myocardial displacements under the supervision of DENSE data. Experimental results demonstrate that one non-limiting proposed model can effectively improve the accuracy of myocardial strain and rotational movements in standard cine CMRs.

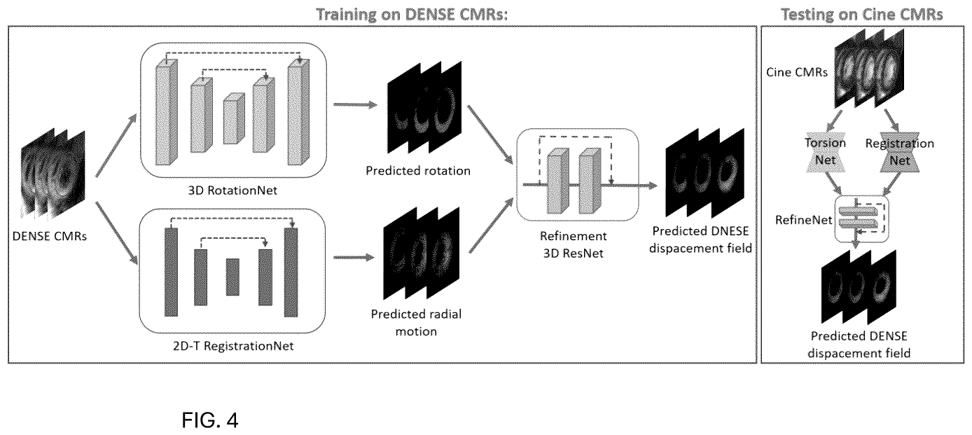

Given a time-sequence of CMR images {I₀, I₁, I₂ . . . , _{T}}, one goal is to predict a sequence of myocardial motions {u¹, u² . . . , u^{T}} from I₀ to {I₁, . . . , I_{T}}. One example of a model comprises three main parts: (i) a rotation estimation sub-network 405 that can capture large degrees of rotational movements across time; (ii) a deformable image registration sub-network 425 to effectively learn radial motions accounted by small rotations; and (iii) a fusion network 450 to integrate the output of the rotation estimation sub-network 405 and deformable image registration sub-networks 425, ultimately predicting the final myocardial displacements. The three parts are trained separately and the overall architecture of an example method is shown in FIG. 4.

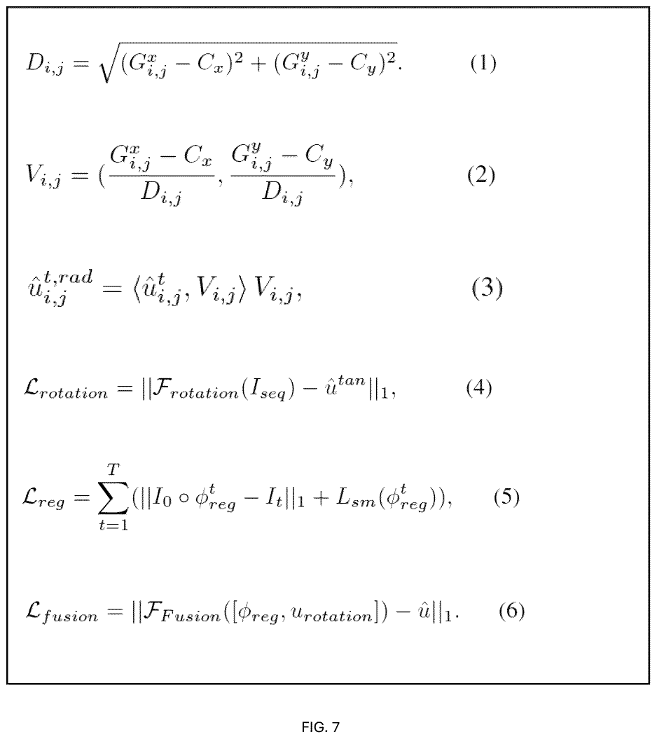

Rotation network. In non-limiting embodiments, this disclosure uses the tangential component of the DENSE displacement, which captures information on large rotations over time, as ground truth to train the proposed rotation network. Consider a sequential 2D DENSE displacement Û∈R^{T×w×h×2}, the DENSE displacement at time t is Û∈R^{T×w×h×2}, defined on the grid (G^{x}, G^{y}), where Gx, Gy∈R^{w×h} and t∈[1, . . . , T]. The center of the grid is located at (Cx, Cy), where Cx=└w/2┘ and Cy=└h/2┘. The distance from grid point (i, j) to the grid center can be computed as Equation 1 of FIG. 7.

Next, the unit radial vector V∈R^{w×h×2} at location (i, j) can be calculated by Equation 2 of FIG. 7.

The radial component of the DENSE displacement at location (i, j) can be obtained by calculating the projection of the displacement field Û (from i, j to t), onto the radial unit vector V i, j, i.e., Equation 3 of FIG. 7, where [symbol] , [symbol] denotes an inner product. The corresponding tangential component can be calculated by Û^{t, tan}=Û^{t}−Û^{t, rad}.

This disclosure employs a 3D UNet [46] to directly capture the rotational movements from the sequential cine MR images and use the decomposed tangential components of the ground truth DENSE displacement fields Û^{tan}∈R^{T×w×h×2} to supervise the output of the rotation network. The loss function can be represented as Equation 4 of FIG. 7, where I_{seq}∈R^{(T+1)×w×h} is the stacked sequential CMR images. Fᵣₒₜₐₜᵢₒₙ (·) is the rotation network and its output is the predicted rotational displacement field uᵣₒₜₐₜᵢₒₙ∈R^{T×w×h×2}.

Registration network. This disclosure employs a deformable image registration network [52] to learn the deformations between the initial frame I₀ and each subsequent frame Iₜ, where t∈[1, . . . , T]. Specifically, this disclosure employs a UNet [53] as the backbone of the registration network to predict the deformation from I₀ to Iₜ. The network is supervised by the loss function of FIG. 7 at Equation 5, where φ (from reg to t)∈R^{w×h×2} denotes the predicted deformation from the registration network, and the symbol (°) denotes the spatial deformation transformation. Lsm (·) is the smooth loss to constrain the smoothness of the displacement field.

Fusion module. When the model gets the myocardial rotation and the registration-based deformation, it further employs a fusion network to predict the final displacement fields. The fusion module is supervised by the ground truth DENSE displacement fields, which can be formulated as Equation 6 of FIG. 7 where φ (reg to t)∈R^{T×w×h×2} is the stacked deformations on the time dimension, [·] is the concatenation operation. F_{Fusion}(·) is the fusion network, which uses the 3D ResNet as the network backbone. The output of the fusion network is the final predicted displacement field.

### 3. EXPERIMENTS

Dataset. This disclosure utilized 741 multiphasic short-axis DENSE slices of the LV, acquired from 284 subjects, including 124 healthy volunteers and 160 patients with various types of heart disease for training [8]. Each DENSE scan was performed in 4 short-axis planes at basal, two mid-ventricular, and apical levels, with a temporal resolution of 17 ms, a pixel size of 2.652 mm2, and a slice thickness of 8 mm. Other parameters included a displacement encoding frequency of 0.1 cycles/mm, a flip angle of 15° (fifteen degrees) and an echo time of 1.08 ms. The model was tested on 105 contoured LV short-axis cine bSSFP images from 40 subjects, including 14 patients and 26 volunteers. All Cine bSSFP images were acquired during repeated breath holds, covering the LV with a temporal resolution of 30-55 ms. Similar to [46, 40], this approach assumes multiphase endocardial and epicardial contours segmented from DENSE and bSSFP images reflect the same underlying cardiac motion. All CMR images underwent temporal and spatial alignment and were cropped to the region of interest with the size of 48×48×T, where T represents the number of temporal frames. This disclosure evaluated the model accuracy on segmental strain analyzed from routine bSSFP CMR with DENSE at matched locations.

Implementation details. The proposed model is implemented in PyTorch and trained on one NVIDIA RTX3090 GPU. This disclosure use the Adam optimizer to optimize the model and set the learning rate to 1×10−4. The model training contains three stages. In the first stage, embodiments train the rotation network using Eq. (4) for 120 epochs. In the second stage, embodiments train a registration network using Eq. (5) to convergence. In the third stage, this disclosure utilizes the registration and rotation networks to train the fusion network using Eq. (6) for another 120 epochs for convergence.

Results. This disclosure compares the proposed method with Strain-Net [48], MotionNet [40], and UNetR [54]. StrainNet and UNetR use the myocardial contours to predict the displacement field of the motion. MotionNet is a simplified version of [40], which employs the registration-based velocity to predict the displacement field. For fair comparison, example embodiments train all methods on the same dataset, and report their best performance.

This disclosure generates strain maps from the predicted motion fields and DENSE ground truth. Since spatial misalignment exists in the myocardium regions between the cine images and the DENSE ground truth, embodiments evaluate the model accuracy on segmental strain. Specifically, this disclosure divides the myocardium in each slice into six segments (inferoseptal, inferior, inferolateral, anterolateral, anterior, anteroseptal), starting from the right ventricle insertion point and proceeding counterclockwise [46]. This disclosure then calculates the average absolute error for strain evaluation in each segment.

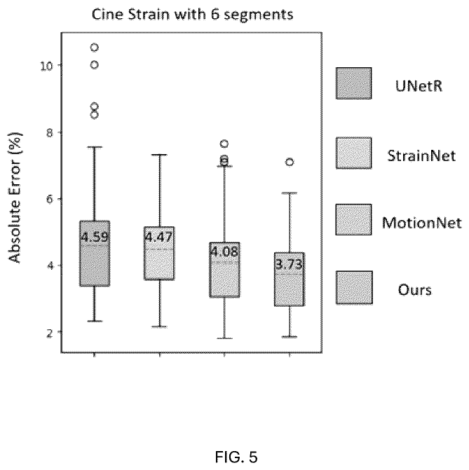

FIG. 5 shows segmental circumferential strain (Ecc) errors of all methods for bSSFP test slices with six segments, using DENSE Ecc as the ground truth. The result of the proposed method shows the smallest error.

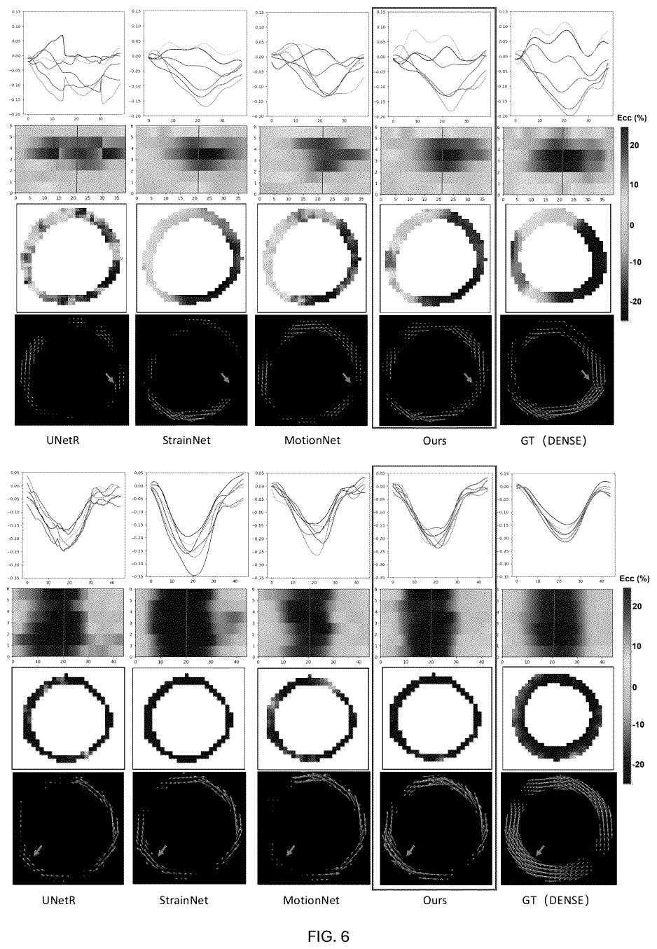

FIG. 6 visualizes two examples of segmental circumferential strain-time curves

, strain-time maps [2], circumferential strain Ecc maps and the estimated rotational movements of all methods. Experimental results show that the proposed method improves the accuracy of myocardial segmental Ecc and rotational movements fairly closer to DENSE.

This disclosure presents a deep learning framework, guided by DENSE ground truth, that incorporates myocardial rotation, an important element of myocardial mechanics (critical for computing torsion) and one that is not well captured by other strain analysis methods that can be applied to routine bSSFP images.

In example embodiments, a computer implemented method trains a neural network to predict a sequence of myocardial motions from cardiac image data of a heart of a subject. The method includes using a computer with computer memory connected to a processor to execute software stored in the computer memory and executing computerized steps. The steps include acquiring a time series of cardiac magnetic resonance (CMR) contour videos of cine data formatted according to displacement encoding with stimulated echoes (DENSE); processing the cine data with a rotation estimation sub-network that models rotational dynamics of a left ventricle of the heart and storing a rotation output in the computer memory; processing the cine data with a radial motion prediction sub-network that performs deformable image registration on the cine data and stores a registration output in the computer memory; applying the rotation output and the registration output to a fusion network that predicts myocardial displacements present in the cine data; and supervising the fusion network with ground truth DENSE data to learn the myocardial displacements for diverse sets of cine data.

The computer implemented method may further include acquiring cine data of displacements of a left ventricle of the heart of the subject. In some embodiments, the software comprises at least one rotation displacement threshold that quantifies myocardial displacement of the left ventricle as large rotations of the left ventricle for rotational displacements above the threshold. Embodiments may include supervising the rotation estimation sub-network with a tangential component of decomposed DENSE ground truth displacements. A three dimensional (3D) U-Net convolutional neural network may be used to capture rotational movements from the cine data and use decomposed tangential components of the ground truth DENSE data to supervise the rotation output. The software may access from memory at least one radial displacement threshold that quantifies myocardial displacement of the left ventricle as minor rotational variations of the left ventricle for radial displacements below the radial displacement threshold. Using the radial motion prediction sub-network allows the neural network to learn radial motions accounted by the minor rotational variations of the left ventricle. The radial motion prediction sub-network may be supervised with a loss function as noted above. The rotation estimation sub-network, radial motion prediction sub-network, and the fusion network are trained separately with respective sets of cine data. The rotation estimation sub-network, radial motion prediction sub-network, and the fusion network are trained separately with same set of cine data.

The term “supervise” as used in this disclosure has the broadest meaning that is commonly accepted in the context of training neural networks with data that has been previously verified (i.e., the neural network can make a comparison between training data and ground truth data to learn functions).

Experimental results demonstrate the effectiveness of the proposed method on myocardial strain prediction. Future work will focus on rigorously assessing the accuracy of torsion measurements and optimizing deep learning from DENSE data to achieve comprehensive and accurate motion/strain analysis of bSSFP images.

The various embodiments described above are provided by way of illustration only and should not be construed to limit the scope of the disclosed technology. Those skilled in the art will readily recognize that various modifications and changes may be made to the disclosed technology without following the example embodiments and implementations illustrated and described herein, and without departing from the spirit and scope of the disclosure and claims here appended. Therefore, other modifications or embodiments as may be suggested by the teachings herein are particularly reserved.

### REFERENCE LIST

1. Keltner JR, Roos MS, Brakeman PR, Budinger TF. Magnetohydrodynamics of blood flow. Magn. Reson. Med. 1990; 16:139-49.
2. Polson MJR, Barker AT, Gardiner S. The effect of rapid rise-time magnetic fields on the ECG of the rat. Clin. Phys. Physiol. Meas. 1982; 3:231-4.
3. Shetty AN. Suppression of Radiofrequency Interference in Cardiac Gated MRI: A Simple Design. Magn. Reson. Med. 1988; 8:84-8.
4. Damji AA, Snyder RE, Ellinger DC, Witkowski FX, Allen PS. RF interference suppression in a cardiac synchronization system operating in a high magnetic field NMR imaging system. Magn. Reson. Imaging. 1988; 6:637-40.
5. Firmin D, Keegan J. Navigator echoes in cardiac magnetic resonance. J. Cardiovasc. Magn. Reson. 2001; 3:183-93.
6. Jung B, Zaitsev M, Hennig J, Markl M. Navigator gated high temporal resolution tissue phase mapping of myocardial motion. Magn. Reson. Med. 2006; 55:937-42.
7. Peters DC, Nezafat R, Eggers H, Stehning C, Manning WJ. 2D free-breathing dual navigator-gated cardiac function validated against the 2D breath-hold acquisition. J. Magn. Reson. Imaging. 2008; 28:773-7.
8. Abd-Elmoniem KZ, Obele CC, Sibley CT, Matta JR, Pettigrew RI, Gharib AM. Free Breathing Single Navigator Gated Cine Cardiac Magnetic Resonance at 3 Tesla: Feasibility Study in Patients. J Comput Assist Tomogr. 2011; 35:382-6.
9. Moghari MH, Barthur A, Amaral ME, Geva T, Powell AJ. Free-breathing whole-heart 3D cine magnetic resonance imaging with prospective respiratory motion compensation. Magn. Reson. Med. [Internet]. 2017; 189:181-9. Available from: http://doi.wiley.com/10.1002/mrm.27021
10. Crowe ME, Larson AC, Zhang Q, Carr J, White RD, Li D, et al. Automated rectilinear self-gated cardiac cine imaging. Magn. Reson. Med. 2004; 52:782-8.
11. Buehrer M, Curcic J, Boesiger P, Kozerke S. Prospective self-gating for simultaneous compensation of cardiac and respiratory motion. Magn. Reson. Med. 2008; 60:683-90.
12. Uribe S, Muthurangu V, Boubertakh R, Schaeffter T, Razavi R, Hill DLG, et al. Whole-heart cine MRI using real-time respiratory self-gating. Magn. Reson. Med. 2007; 57:606-13.
13. Uribe S, Beerbaum P, Sørensen TS, Rasmusson A, Razavi R, Schaeffter T. Four-dimensional (4D) flow of the whole heart and great vessels using real-time respiratory self-gating. Magn. Reson. Med. 2009; 62:984-92.
14. Feng X, Salerno M, Kramer CM, Meyer CH. Non-Cartesian balanced steady-state free precession pulse sequences for real-time cardiac MRI. Magn. Reson. Med. 2016; 75:1546-55.
15. Kim D, Gilson WD, Kramer CM, Epstein FH. Myocardial tissue tracking with two-dimensional cine displacement-encoded MR imaging: development and initial evaluation. Radiology 2004; 230(3):862-871.
16. Zhong X, Spottiswoode BS, Meyer CH, Kramer CM, Epstein FH. Imaging three-dimensional myocardial mechanics using navigator-gated volumetric spiral cine DENSE MRI. Magnetic resonance in medicine 2010; 64(4):1089-1097.
17. Young AA, Li B, Kirton RS, Cowan BR. Generalized spatiotemporal myocardial strain analysis for DENSE and SPAMM imaging. Magnetic resonance in medicine. 2012; 67(6):1590-9.
Lin K, Meng L, Collins JD, Chowdhary V, Markl M, Carr JC. Reproducibility of cine displacement encoding with stimulated echoes (DENSE) in human subjects. Magnetic resonance imaging. 2017; 35:148-53.
18. Gilliam AD, Epstein FH. Automated motion estimation for 2-D cine DENSE MRI. IEEE transactions on medical imaging. 2012; 31(9):1669-81.
19. Auger DA, Bilchick KC, Gonzalez JA, Cui SX, Holmes JW, Kramer CM, et al. Imaging left-ventricular mechanical activation in heart failure patients using cine DENSE MRI: Validation and implications for cardiac resynchronization therapy. Journal of Magnetic Resonance Imaging. 2017.
20. Mangion K, Carrick D, Carberry J, Mahrous A, McComb C, Gao H, et al. Comparative prognostic value of myocardial strain derived from DENSE CMR: the British Heart Foundation MR-MI study. The Lancet. 2017; 389:S66.
21. Jing L, Binkley CM, Suever JD, Umasankar N, Haggerty CM, Rich J, et al. Cardiac remodeling and dysfunction in childhood obesity: a cardiovascular magnetic resonance study. Journal of Cardiovascular Magnetic Resonance. 2016; 18(1):28.
22. Ehman RL, Felmlee JP. Adaptive technique for high-definition MR imaging of moving structures. Radiology. 1989; 173(1):255-63.
23. Larson AC, White RD, Laub G, McVeigh ER, Li D, Simonetti OP. Self-gated cardiac cine MRI. Magnetic Resonance in Medicine. 2004; 51(1):93-102.
24. Uribe S, Muthurangu V, Boubertakh R, Schaeffter T, Razavi R, Hill DL, et al. Whole-heart cine MRI using real-time respiratory self-gating. Magnetic Resonance in Medicine. 2007; 57(3):606-13.
25. Usman M, Atkinson D, Odille F, Kolbitsch C, Vaillant G, Schaeffter T, et al. Motion corrected compressed sensing for free-breathing dynamic cardiac MRI. Magnetic resonance in medicine. 2013; 70(2):504-16.
26. Kellman P, Larson AC, Hsu LY, Chung YC, Simonetti OP, McVeigh ER, et al. Motion-corrected free-breathing delayed enhancement imaging of myocardial infarction. Magnetic resonance in medicine. 2005; 53(1):194-200.

27. Stehning C, Börnert P, Nehrke K, Eggers H, Stuber M. Free-breathing whole-heart coronary MRA with 3D radial SSFP and self-navigated image reconstruction. Magnetic resonance in medicine. 2005; 54(2):476-80.
28. Henningsson M, Koken P, Stehning C, Razavi R, Prieto C, Botnar RM. Whole-heart coronary MR angiography with 2D self-navigated image reconstruction. Magnetic resonance in medicine. 2012; 67(2):437-45.
29. Lai P, Larson AC, Bi X, Jerecic R, Li D. A dual-projection respiratory self-gating technique for whole-heart coronary MRA. Journal of Magnetic Resonance Imaging. 2008; 28(3):612-20.
30. Chow K, Yang Y, Shaw P, Kramer CM, Salerno M. Robust free-breathing SASHA T 1 mapping with high-contrast image registration. Journal of Cardiovascular Magnetic Resonance. 2016; 18(1):47.
31. Zhong X, Spottiswoode BS, Cowart EA, Gilson WD, Epstein FH. Selective suppression of artifact-generating echoes in cine DENSE using through-plane dephasing. Magnetic resonance in medicine. 2006; 56(5):1126-31.
32. Mills P, Chew W, Litt L, Moseley M. Localized imaging using stimulated echoes. Magnetic resonance in medicine 1987; 5(4):384-389.
32. Gudbjartsson H, Patz S. The Rician distribution of noisy MRI data. Magnetic resonance in medicine 1995; 34(6):910-914.
33. Goto Y, Ishida M, Takase S, Sigfridsson A, Uno M, Nagata M, et al. Comparison of Displacement Encoding With Stimulated Echoes to Magnetic Resonance Feature Tracking for the Assessment of Myocardial Strain in Patients With Acute Myocardial Infarction. The American Journal of Cardiology. 2017; 119(10):1542-7.
34. Kihlberg J, Haraldsson H, Sigfridsson A, Ebbers T, Engvall JE. Clinical experience of strain imaging using DENSE for detecting infarcted cardiac segments. Journal of Cardiovascular Magnetic Resonance. 2015; 17(1):50.
35. Winkelmann S, Schaeffter T, Koehler T, Eggers H, Doessel O. An optimal radial profile order based on the Golden Ratio for time-resolved MRI. IEEE transactions on medical imaging. 2007; 26(1):68-76.
36. Kim YC, Narayanan SS, Nayak KS. Flexible retrospective selection of temporal resolution in real-time speech MRI using a golden-ratio spiral view order. Magnetic resonance in medicine. 2011; 65(5):1365-71.
37. Aletras AH, Balaban RS, Wen H. High-resolution strain analysis of the human heart with fast-DENSE. Journal of Magnetic Resonance. 1999; 140(1):41-57.
38. Çiçek, Özgün et al., 3D U-Net: Learning Dense Volumetric Segmentation from Sparse Annotation published in MICCAI 2016.
39. K. C. Bilchick, D. A. Auger, M. Abdishektaei, R. Mathew, M.-W. Sohn, X. Cai, C. Sun, A. Narayan, R. Malhotra, A. Darby, et al., “Cmr dense and the seattle heart failure model inform survival and arrhythmia risk after crt,” Cardiovascular Imaging, vol. 13, no. 4, pp. 924-936, 2020.
40. J. Xing, N. Wu, K. C. Bilchick, F. H. Epstein, and M. Zhang, “Multimodal learning to improve cardiac late mechanical activation detection from cine mr images,” in 2024 IEEE International Symposium on Biomedical Imaging (ISBI). IEEE, 2024, pp. 1-4.
41. K. Mangion, D. Carrick, J. Carberry, A. Mahrous, C. McComb, K. G. Oldroyd, H. Eteiba, M. Lindsay, M. McEntegart, S. Hood, et al., “Circumferential strain predicts major adverse cardiovascular events following an acute st-segment-elevation myocardial infarction,” Radiology, vol. 290, no. 2, pp. 329-337, 2019.
42. L. Ernande, H. Thibault, C. Bergerot, P. Moulin, H. Wen, G. Derumeaux, and P. Croisille, Systolic myocardial dysfunction in patients with type 2 diabetes mellitus: identification at mr imaging with cine displacement encoding with stimulated echoes,” Radiology, vol. 265, no. 2, pp. 402-409, 2012.
43. M. Qiao, Y. Wang, Y. Guo, L. Huang, L. Xia, and Q. Tao, “Temporally coherent cardiac motion tracking from cine mri: Traditional registration method and modern cnn method,” Medical Physics, vol. 47, no. 9, pp. 4189-4198, 2020.
44. C. Qin, W. Bai, J. Schlemper, S. E. Petersen, S. K. Piechnik, S. Neubauer, and D. Rueckert, “Joint motion estimation and segmentation from undersampled cardiac mr image,” in Machine Learning for Medical Image Reconstruction. Springer, 2018, pp. 55-63.
45. M. S. Amzulescu, M. De Craene, H. Langet, A. Pasquet, D. Vancraeynest, A.-C. Pouleur, J.-L. Vanoverschelde, and B. Gerber, “Myocardial strain imaging: review of general principles, validation, and sources of discrepancies,” European Heart Journal-Cardiovascular Imaging, vol. 20, no. 6, pp. 605-619, 2019.
46. Y. Wang, C. Sun, S. Ghadimi, D. C. Auger, P. Croisille, M. Viallon, K. Mangion, C. Berry, C. M. Haggerty, L. Jing, et al., “Strainnet: improved myocardial strain analysis of cine mri by deep learning from dense,” Radiology: Cardiothoracic Imaging, vol. 5, no. 3, pp. e220196, 2023.
47. J. Xing, N. Jayakumar, N. Wu, Y. Wang, F. H. Epstein, and M. Zhang, “Lamod: Latent motion diffusion model for myocardial strain generation,” arXiv preprint arXiv:2407.02229, 2024.
48. I. K. R\ddot{ }ussel, M. J. G\ddot{ }otte, J. G. Bronzwaer, P. Knaapen, W. J. Paulus, and A. C. van Rossum, “Left ventricular torsion: an expanding role in the analysis of myocardial dysfunction,” JACC: Cardiovascular Imaging, vol. 2, no. 5, pp. 648-655, 2009.
49. I. K. R\ddot{ }ussel, M. J. G\ddot{ }otte, J. G. Bronzwaer, P. Knaapen, W. J. Paulus, and A. C. van Rossum, “Left ventricular torsion: an expanding role in the analysis of myocardial dysfunction,” JACC: Cardiovascular Imaging, vol. 2, no. 5, pp. 648-655, 2009.
50. A. M. S. Omar, S. Vallabhajosyula, and P. P. Sengupta, “Left ventricular twist and torsion: research observations and clinical applications,” Circulation: Cardiovascular Imaging, vol. 8, no. 6, pp. e003029, 2015.
51. X. Zhong, B. S. Spottiswoode, C. H. Meyer, C. M. Kramer, and F. H. Epstein, “Imaging three-dimensional myocardial mechanics using navigator-gated volumetric spiral cine dense mri,” Magnetic resonance in medicine, vol. 64, no. 4, pp. 1089-1097, 2010.
52. N. Wu, J. Xing, and M. Zhang, “Tlrn: Temporal latent residual networks for large deformation image registration,” in International Conference on Medical Image Computing and Computer-Assisted Intervention. Springer, 2024, pp. 728-738.
53. O. Ronneberger, P. Fischer, and T. Brox, “U-net: Convolutional networks for biomedical image segmentation,” in Medical image computing and computer assisted intervention—MICCAI 2015: 18th international conference, Munich, Germany, October 5-9, 2015, proceedings, part III 18. Springer, 2015, pp. 234-241.
54. A. Hatamizadeh, Y. Tang, V. Nath, D. Yang, A. Myronenko, B. Landman, H. R. Roth, and D. Xu, “Unetr: Transformers for 3d medical image segmentation,” in Proceedings of the IEEE/CVF winter conference on applications of computer vision, 2022, pp. 574-584.

What is claimed is:

1. A computer implemented method to train a neural network to predict a sequence of myocardial motions from cardiac image data of a heart of a subject, comprising:
using a computer with computer memory connected to a processor to execute software stored in the computer memory and executing computerized steps comprising:
acquiring a time series of cardiac magnetic resonance (CMR) contour videos of cine data formatted according to displacement encoding with stimulated echoes (DENSE);
processing the cine data with a rotation estimation sub-network that models rotational dynamics of a left ventricle of the heart and storing a rotation output in the computer memory;
processing the cine data with a radial motion prediction sub-network that performs deformable image registration on the cine data and stores a registration output in the computer memory;
applying the rotation output and the registration output to a fusion network that predicts myocardial displacements present in the cine data;
supervising the fusion network with ground truth DENSE data to learn the myocardial displacements for diverse sets of cine data.

2. The computer implemented method of claim 1, further comprising acquiring cine data of displacements of a left ventricle of the heart of the subject.

3. The computer implemented method of claim 2, wherein the software comprises at least one rotation displacement threshold that quantifies myocardial displacement of the left ventricle as large rotations of the left ventricle for rotational displacements above the threshold.

4. The computer implemented method of claim 1, further comprising supervising the rotation estimation sub-network with a tangential component of decomposed DENSE ground truth displacements.

5. The computer implemented method of claim 4, further comprising utilizing a three dimensional (3D) U-Net convolutional neural network to capture rotational movements from the cine data and use decomposed tangential components of the ground truth DENSE data to supervise the rotation output.

6. The computer implemented method of claim 1 wherein the software comprises at least one radial displacement threshold that quantifies myocardial displacement of the left ventricle as minor rotational variations of the left ventricle for radial displacements below the radial displacement threshold.

7. The computer implemented method of claim 6, further comprising using the radial motion prediction sub-network to learn radial motions accounted by the minor rotational variations of the left ventricle.

8. The computer implemented method of claim 7, further comprising supervising the radial motion prediction sub-network with a loss function.

9. The computer implemented method of claim 1, wherein the rotation estimation sub-network, radial motion prediction sub-network, and the fusion network are trained separately with respective sets of cine data.

10. The computer implemented method of claim 1, wherein the rotation estimation sub-network, radial motion prediction sub-network, and the fusion network are trained separately with same set of cine data.
